(19) United States
(12) Patent Application Publication
(10) Pub. No.: US 2005/0148283 A1
(43) Pub. Date: Jul. 7, 2005

(54) Interactive display

(76) Inventor: Norman David Schwalm, Bergenfield, NJ (US)

Correspondence Address: LACKENBACH SIEGEL, LLP, LACKENBACH SIEGEL BUILDING, 1 CHASE ROAD, SCARSDALE, NY, 10583, US

(21) Appl. No.: 11/027,882

(22) Filed: Dec. 30, 2004

Related U.S. Application Data
(60) Provisional application No. 60/534,328, filed on Jan. 5, 2004.
(60) Provisional application No. 60/535,087, filed on Jan. 8, 2004.

(51) Int. Cl. G09B 23/28; A61B 5/00; F21S 13/02; A63H 33/26; F21V 1/00; A61B 5/02; F21S 13/12; F21S 2/00

(52) U.S. Cl. 446/485; 434/272; 362/457; 362/458

(57) ABSTRACT

The invention provides a novelty item for displaying functioning of a heart, comprising a body organ, a pulsating light emitter aimed to approximately correspond to the pulsation of the human heart, and an electric/electronic circuit to vary at least the rate of pulsating light in accordance with signals received by the circuit.

### FIELD OF THE INVENTION

The present invention relates to novelty items. In particular, the invention relates to a novelty interactive display for entertainment and/or mood modifying, and/or therapeutic item in the form of a human body or part of a body, including a stylized heart for displaying functioning of the “heartbeat”. Specifically, both the pulsating rate and the intensity of the displayed “heartbeat” can be varied in response to inputs from various sensors connected to the electronics controlling the item.

### BACKGROUND OF THE INVENTION

The toy or novelty industry has produced many forms of representations of the human heart, either separately or as combined in numerous forms, e.g., embodied in dolls or other toys, as stand-alone novelties, on bracelets and necklaces, as watches or clocks. Prior art also includes models of beating hearts, using pumps, servos, electric motors and other devices to simulate the actual beating of a heart. Other prior art includes various heart-shaped devices with varying light-emission properties and switching mechanisms, some accompanied by sound simulating the human heartbeat. To the inventor's knowledge, there has been no prior art that includes variability in rate and intensity of light emission as dependent upon one or a combination of environmental variables including ambient light, heat, motion, and proximity of an external stimulus.

In addition, medical heart models are known for purposes of teaching medical staff or demonstrating modes of heart functions and heart failure, as seen in U.S. Pat. No. 5,482,472 to Garoni et al., U.S. Pat. No. 6,353,151 to Leinwand et al. and U.S. Pat. No. 6,685,481 to Chamberlain. Such models are, of course, not intended for use as novelty entertainment, or mood-modifying or therapeutic items, and are far too expensive for this purpose.

### DISCLOSURE OF THE INVENTION

It is therefore one of the objects of the present invention to obviate the limitations of prior art heart models and to provide a reactive living body organ which may be used for entertainment and/or mood-modifying, and/or therapeutic purposes.

It is a further object of the present invention to provide a body organ including at least one sensor for detecting the presence of a nearby living body, usually a person, and reacting thereto by changes in light pulsating rate and/or intensity, and/or color.

The term ‘body organ’ as used herein is meant to define any part of a human or a non-human living body, which preferably, but not necessarily, includes or is composed of a configuration of a heart. This configuration may be a head or face resembling a human or non-human head, a heart, a transparent upper torso of a human body, including a heart therein.

The present invention achieves the above objects by providing a novelty item for displaying functioning of a heart, comprising a body organ, a pulsating light emitter aimed to approximately correspond to the pulsation of the human heart, and an electric/electronic circuit to vary at least the rate of pulsating light in accordance with signals received by said circuit.

In a preferred embodiment of the present invention there is provided a novelty item, further provided with a proximity sensor, the output of which is fed into an electric/electronic circuit to modify at least the intensity or the pulsating rate of the light emitter, when an approaching living body is detected by the sensor.

In yet a further preferred embodiment of the invention wherein the novelty item is provided with a sensor for detecting and electronically recording the pulse rate of a living body limb placed into contact with the sensor, the electronic representation of the pulse beat being transferred to the electric/electronic circuit, and the circuit being arranged to adjust the pulsating rate of the emitted light in accordance with the electronic representation of the pulse beat.

In a further embodiment of the present invention there is provided a heart-like item provided with an input device allowing a user to insert data relating to the mood of a user, the input being operatively connected to the electric/electronic circuit which adjusts the intensity and pulsating rate of the emitted light according to values stored in a memory.

It will thus be realized that the novel item of the present invention serves to provide much entertainment due to the unexpectedness of its various reactions to people in its vicinity. These reactions can result from varying light levels, pulse sensor, motion detectors, voice sensors reacting to, e.g., recitation of a word or tune or further items detected by sensors in the model.

It will also be evident that the model can be constructed in various sizes, for example miniature for use of one person or jumbo size entertaining a group or audience.

### BRIEF DESCRIPTION OF THE DRAWINGS

The invention will now be described in connection with certain preferred embodiments with reference to the following illustrative figures, so that it may be more fully understood.

With specific reference now to the figures in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagrammatic view of a preferred embodiment of the novel item according to the present invention;

FIG. 2 is a diagram of an anatomical configuration of a heart;

FIG. 3 is a diagrammatic view of a preferred embodiment of the novel item housed in a transparent display case;

FIG. 4 is a diagrammatic view of a preferred embodiment of the novel item powered by solar cells;

FIG. 5 is a diagrammatic view of a preferred embodiment of the novel item arranged to respond to changes in ambient light;

FIG. 6 is a diagrammatic view of a preferred embodiment of the novel item including a user input device;

FIG. 7 is a diagrammatic view of a preferred embodiment of the novel item responsive to the heartbeat of a user;

FIG. 8 is a diagrammatic view of a preferred embodiment of the novel item responsive to audio inputs;

FIGS. 9 and 10 are graphical representations of light emissions at different pulsating rates and intensities;

FIG. 11 illustrates a further embodiment of the invention, and

FIG. 12 illustrates a communication system utilizing the embodiment of FIG. 11.

### DETAILED DESCRIPTION OF PREFERRED EMBODIMENTS

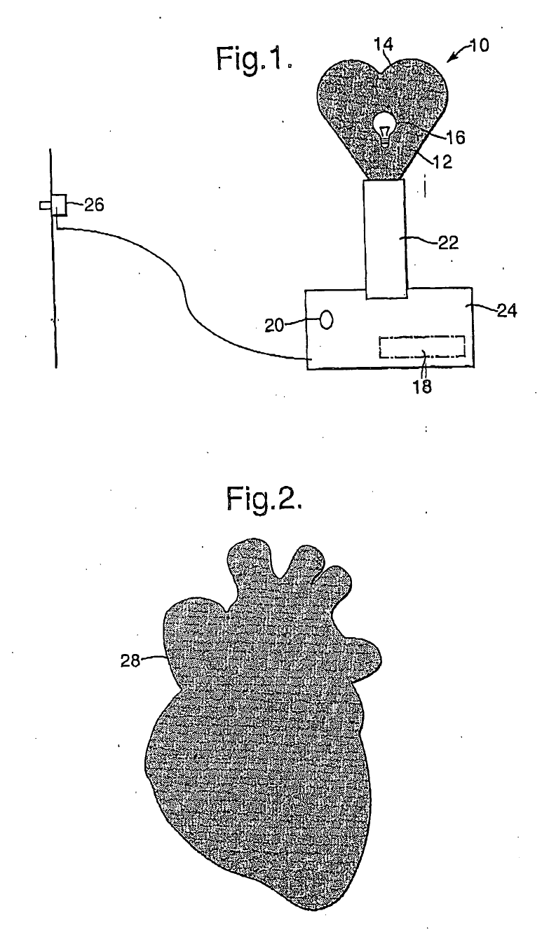

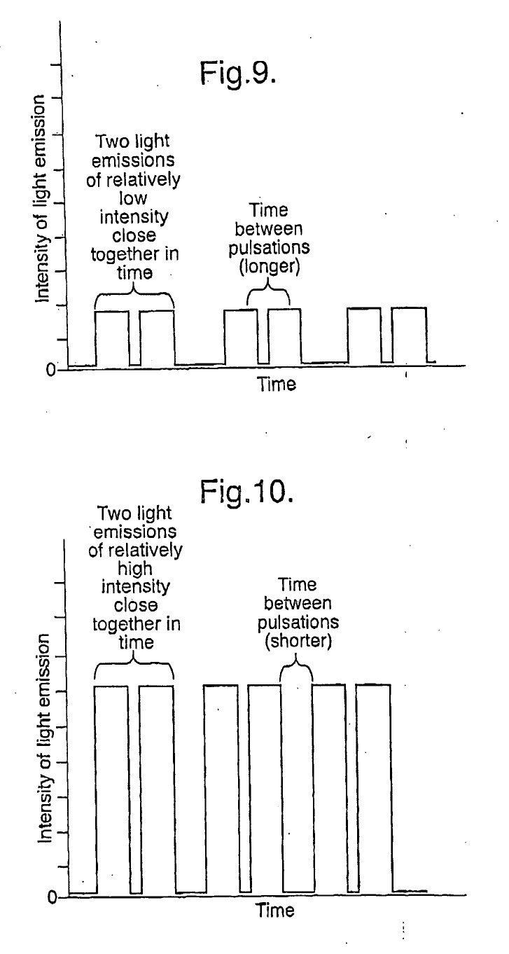

There is seen in FIG. 1 a novelty item 10 displaying a body organ 12, e.g., a heart shaped body. In the shown embodiment, the heart-shaped body is of a stylized form, with a single curved depression 14 on its upper edge and a tapered lower section. The body organ 12 comprises an enclosure suitably made of e.g., a translucent material, and contains a pulsating variable-intensity light emitter 16. The light pulsations to be described hereinafter with reference to FIGS. 9 and 10, are timed to approximately correspond to the normal pulsation rate of the human heart, unless signals are received from an input to alter the timing and/or the light intensity, and/or the color of light. Electric/electronic circuitry 18 including a microprocessor, is provided to vary the timing and intensity of the pulsating light in accordance with signals transmitted to the circuitry.

In the present embodiment, input is provided by a proximity sensor 20, such as an infrared sensor responding to nearby effects generated by human bodies. The sensors may have the capability of detecting from a relatively far distance or only from a nearby location. Sensor output is fed into the electric/electronic circuit 18 to increase the intensity and/or the pulsating rate of the emitted light when an approaching person is detected by the sensor 20. There may be provided more than a single sensor, e.g., a near-detect sensor and a far-detect sensor, each capable of being operated separately.

Advantageously, the body organ 12 is mounted on a pedestal 22, the base 24 of which houses the electronic circuitry 18.

Power is supplied in the present embodiment, by a transformer-rectifier 26 plugged into a line socket. In alternative embodiments, power can be supplied from a wall socket, from a battery or a rechargeable battery, and/or from solar cells.

Referring now to FIG. 2, there is illustrated a configuration of a heart-shaped body 28 of anatomically more realistic configuration. Such configuration can be substituted for the stylized shape seen in FIG. 1.

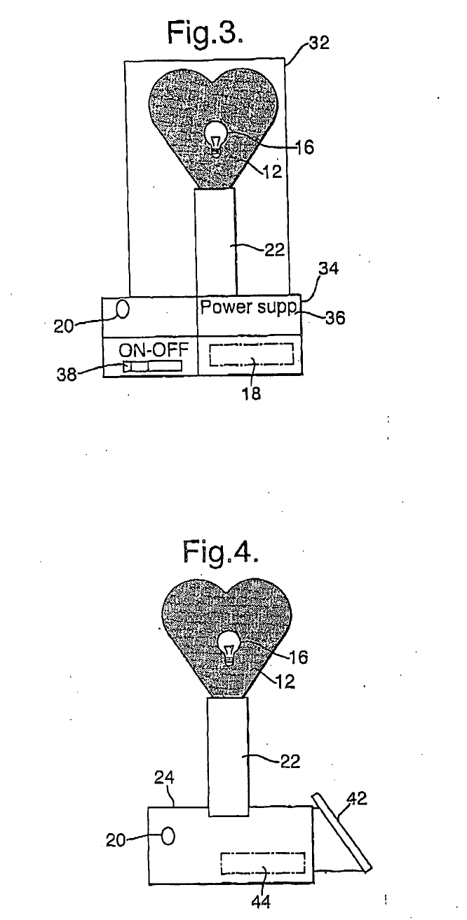

FIG. 3 illustrates a heart-shaped body 12 housed in a transparent display case 32. The case is suitably made of an acrylic polymer. Where the heart-shaped body is subjected to possible vandalism the display case may be made of a polycarbonate. Preferably the case 32 is supported on an opaque stand 34. The stand is utilized to store a power supply 36, which may comprise a rechargeable electric battery and an ON-OFF switch 38. The switch 38 is advantageously a 3-way switch having three positions, which control the following states:
Position A—OFF—The device is off. Power to the device is disconnected.
Position B—ON—The device is on and is activated by either ‘far-detect’ sensors or ‘near-detect’ proximity detectors. The device will not be actuated unless a ‘far-detect’ signal or a ‘near-detect’ signal is received by electrical/electronic circuit 18.
Position C—ON—The device is on with heart rate and/or intensity determined by ambient light levels. Both ‘far-detect’ and ‘near-detect’ behave as above.

Minimal predetermined light level is required for the device to operate in positions B and C, namely, if minimal light level as set does not exist, the device will not react to the proximity level signals.

Seen in FIG. 4 is the novelty item 10, wherein the power supply comprises a group of solar cells 42, electrically connected to the power supply 44.

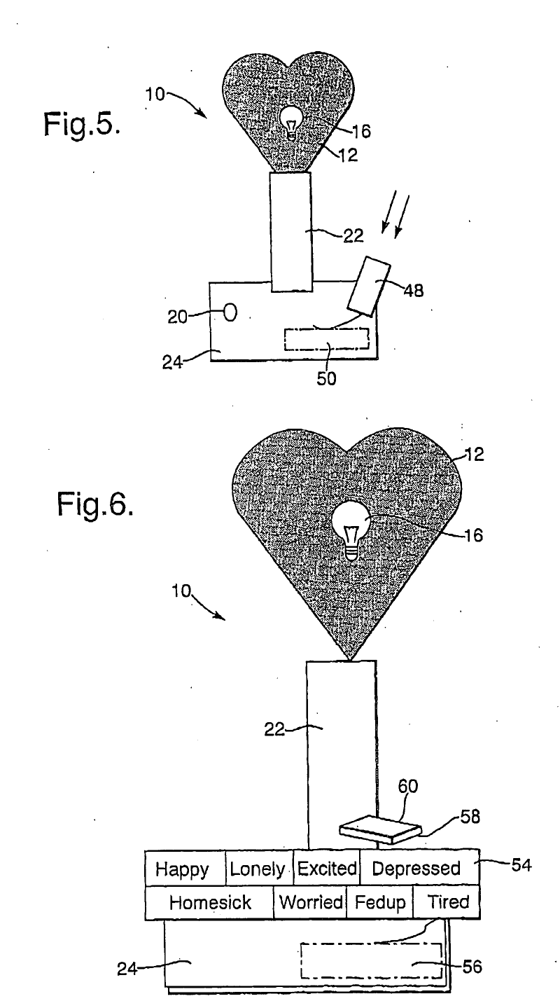

Referring now to FIG. 5, there is depicted an embodiment of the novelty item 10 provided with an ambient light measurement photocell 48. The output of the photocell 48 is fed into the electronic circuitry 50 to increase the intensity of the emitted light under conditions of high ambient light and to reduce the intensity thereof under conditions of low ambient light.

FIG. 6 shows an embodiment of the novelty item 10, further provided with an input device 54 allowing a user to insert data relating to his/her mood. The input device shown offers users to press keys marked, e.g., Happy, Lonely, Excited, Depressed, Homesick, Worried, etc. The keys are operatively connected to the electronic circuitry 56, which adjusts the intensity and pulsating rate of the emitted light according to values stored in a memory look-up table. Optionally, there is provided a recess 58 containing leaflets 60 bearing a text relating the emotional state of a user to the functioning of human heart.

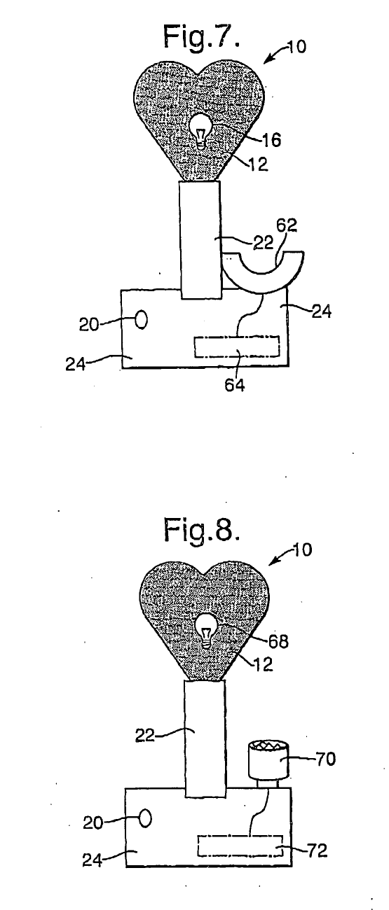

FIG. 7 illustrates a novelty item 10, further provided with a sensor 62 for detecting and electronically recording the pulse beat of a living body limb, e.g., a palm, a finger or a wrist. The limb is placed into contact with the suitably configured sensor 62, and the electronic representation of the pulse beat is transferred to the electronic circuitry 64. The circuitry 64 is programmed to adjust the pulsating rate of the emitted light in accordance with the received electronic representation of the pulse beat.

Seen in FIG. 8 is a novelty item 10 according to the present invention, wherein the light emitter 68 is normally in an OFF state. The item 10 is further provided with a microphone 70 connected to sound recognition software, which is part of the electronic circuitry 72 holding electronic representations of sounds in its memory. The light emitter 68 is switched to an ON state by the circuitry 72 when representation of sounds electronically stored in the circuitry memory substantially match representation of sounds electronically received from the microphone 70. Thus the item 10 can be programmed to respond to any word, phrase or melody.

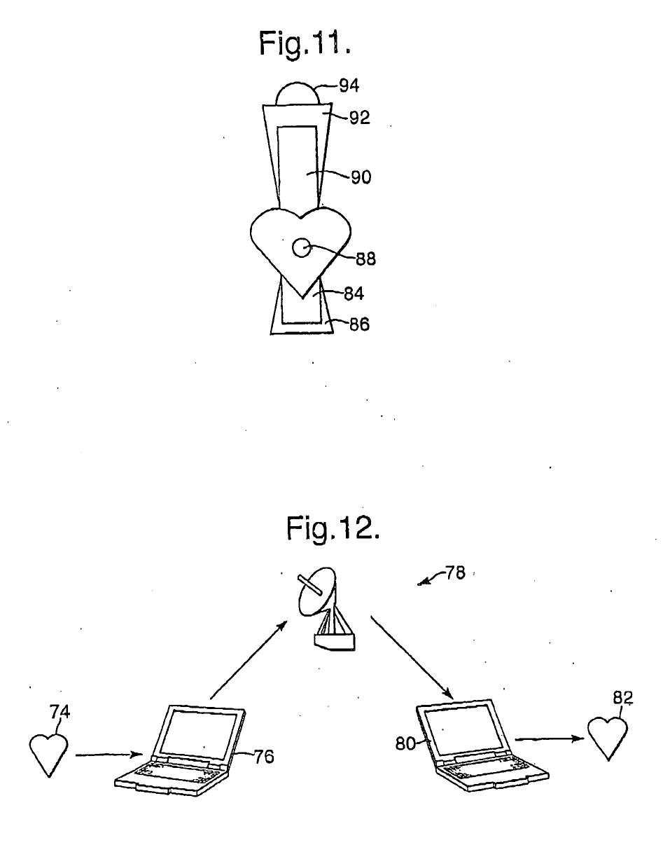

Turning now to FIGS. 9 and 10, the diagrams illustrate how both intensity and pulsating rate of the light can be varied. The two qualities are substantially independent, i.e., each can be varied independently of the other.

Whereas the embodiments of FIGS. 1 to 8 were mainly directed to novelty items which are stationary in the sense that they are placed on a table, shelf or the like, FIG. 11 illustrates an embodiment wherein the item 10 is mobile and/or wearable. Such a mobile and/or wearable item 10 may be constituted by a bracelet, a ring, a pin-like unit, a pendant, etc. or be packaged in a configuration similar to a cell-phone, suitable for carrying in a pocket or pouch.

Referring now to FIG. 12, the item 10 with a sending unit 74, also includes a software which contains a proprietary code allowing it to identify itself to a local computer 76, via proprietary software hosted on the Internet 78, either as a stand-alone web site or using current messaging services. The software will identify only a unit with the proprietary code assigned to it and allow communications from the sending unit 74 to a computer 76, only after proper identification has been established. Once identified, the sending unit 74 transmits a signal/event (the beat pattern, frequency and intensity, color, etc.) generated by it to the local computer 76 via one or more computer technologies (e.g., USB, and/or serial port, and/or IR data port, and/or RF). That event is then translated into the proper form required by the proprietary software to initiate an event at a remotely located computer 80. The remote computer 80 receives the event upon the remote computer being identified by its own proprietary code. Once identified and having received the event, the remote computer 80 then, in turn, outputs the appropriate signal/event via one or more of the above-described technologies, to the receiving unit 82, which then displays the same beat pattern, frequency and intensity as displayed by the original (local) unit 74.

In this manner, a person on one end of the globe can initiate a unit's response and can transmit that same response to a person on the other end of the globe, who, by virtue of his/her own unit, can see the response of the sending unit on his/her own unit. This can be done either in real-time or via stored files, at the user's request, via the sending/receiving computer's software settings.

The same communication protocols can be used to send predefined emotional heartbeat parameters, e.g., happy, sad, surprised, excited, aroused, etc.) via proprietary software resident at the heartbeat web site. Alternatively, the user may set heartbeat parameters via the software itself, bypassing the physical unit, and may send those to a remote computer, as described above. It should be noted, however, that access to the software that allows these parameters to be manipulated is only available if the user owns and has “connected” an original item 10 in any of its embodiments to the originating computer.

FIG. 11 specifically illustrates a wearable down- and up-loadable heartbeat unit 74, which is portable, miniature, includes the proprietary software identification code that allows communication to a computer and access to the web-based software, and which contains a standard USB connection 84 and a connector cover 86. The unit is capable of downloading signals of heartbeat parameters being communicated to it from a sending unit via a computer, and displaying those parameters via miniature colored LED 88 of the wearable unit, after downloading. Thus, a remote user who has access to heart software 90 may send selected or assigned heartbeat parameters to a local computer via standard communication protocols, which will be downloaded to a receiving wearable unit, and displayed via that unit itself, while it is worn.

Alternatively, one may access the software and modify such parameters locally and then download directly. That is to say, one may decide that the user wants to display a “happy” heartbeat at any particular time, select or generate the parameters on the user's own local computer and download them to the unit for display, while wearing the item. Also seen in FIG. 11 is an outer housing 92 and a necklace ring 94 for affixing the unit to any suitable place.

It will be evident to those skilled in the art that the invention is not limited to the details of the foregoing illustrated embodiments and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

1. A novelty item for displaying functioning of a heart, comprising a body organ, a pulsating light emitter aimed to approximately correspond to the pulsation of the human heart, and an electric/electronic circuit to vary at least the rate of pulsating light in accordance with signals received by said circuit.

2. The item according to claim 1, wherein said body organ is supported on a stand.

3. The item according to claim 1, wherein said body organ is a heart-shaped body configured to form an anatomically realistic shape.

4. The item according to claim 1, wherein said body organ is a heart-shaped body of stylized form.

5. The item according to claim 1, wherein said body organ is made of translucent material.

6. The item according to claim 5, wherein said pulsating light emitter is disposed inside said body organ.

7. The item according to claim 1, further comprising a transparent display case.

8. The item according to claim 1, further comprising a power supply.

9. The item according to claim 8, wherein said power supply is a rechargeable battery.

10. The item according to claim 8, wherein said power supply is a panel of solar cells.

11. The item according to claim 1, further comprising at least one proximity sensor, the output of which is fed into said electric/electronic circuit to modify the intensity and/or color, and or pulsating rate of the light, when an approaching person is detected by said sensor.

12. The item according to claim 1, further comprising an ambient light measurement photocell, the output of which is fed into said electric/electronic circuit to increase the intensity of said emitted light under conditions of high ambient light and to reduce said intensity under conditions of low ambient light.

13. The item according to claim 1, further comprising an input device allowing a user to input signals relating to a mood of the user, said input being operatively connected to the electric/electronic circuit for adjusting the intensity and/or color, and/or pulsating rate of the emitted light.

14. The item according to claim 1, further comprising a further sensor for detecting and electronically recording the pulse beat of a living body limb placed into contact with said further sensor, the electronic representation of the pulse beat being transferred to said electric/electronic circuit, and said circuitry being arranged to adjust the pulsating rate of the emitted light in accordance with the electronic representation of said pulse beat.

15. The item according to claim 1, wherein said light emitter is normally in an OFF state, said item further comprising a microphone connected to sound recognition software connected to said electric/electronic circuit storing sounds in its memory, wherein said light emitter is switched to an ON state by said circuitry when representation of sounds electronically stored in said memory, substantially match representation of sounds electronically received from said microphone.

16. The item according to claim 1, further comprising a 3-position switch controlling three states, an ON-OFF state, a ‘near-detect’ or ‘far-detect’ state and/or an ambient light level, light emitter intensity-dependent state.

17. The item according to claim 1, in combination with a leaflet relating the emotional state of a user to the intensity and pulsating rate of said emitted light.

18. The item according to claim 1, further comprising an identification code allowing communication with a computer and access to a web-based software.

19. The item according to claim 1, further comprising a standard USB connection for downloading signals from a computer.

20. The item according to claim 1, configured as a compact wearable unit.
